(19) United States
(12) Patent Application Publication
(10) Pub. No.: US 2023/0301516 A1
(43) Pub. Date: Sep. 28, 2023

(54) Care System for Elderly and Physically Disabled Persons

(71) Applicants: Maria Dimbla, San Jose, CA (US); Marie Aspiras, San Jose, CA (US)

(72) Inventors: Maria Dimbla, San Jose, CA (US); Marie Aspiras, San Jose, CA (US)

(21) Appl. No.: 18/186,436

(22) Filed: Mar. 20, 2023

Related U.S. Application Data
(60) Provisional application No. 63/322,838, filed on Mar. 23, 2022.

(51) Int. Cl. A61B 5/00; A61B 5/11; G16H 40/20; G16H 80/00

(52) U.S. Cl. CPC A61B 5/0022; A61B 5/1112; G16H 40/20; G16H 80/00

(57) ABSTRACT

The present invention is a medical care providing system for improving care quality for elderly and physically disabled persons/patients. The system uses a computer-implemented application installed in user devices of persons/patients (care seekers) enabling the persons/patients to request for one or more of jobs, medications, consultations, care visits, wheelchairs, gurneys, ambulances, and more. The system automatically assigns a care giver, a doctor, a pharmacy, a visit, and more for servicing the request of the care seeker. Automatic assignment may be done based on location, profile, preferences, and more of the care seeker and real time notifications are provided to the care seeker. The application enables the care seeker to connect video calls and audio calls with interviews for jobs, consultations with doctors, et.al. In some embodiments, the system uses GPS for detecting locations of the care seeker, care giver, and other entities.

### CROSS-REFERENCE TO RELATED APPLICATION

The present application claims priority to, and the benefit of, U.S. Provisional Application No. 63/322,838, which was filed on Mar. 23, 2022, and is incorporated herein by reference in its entirety.

### FIELD OF THE INVENTION

The present invention relates generally to the field of healthcare systems. More specifically, the present invention relates to a novel medical care system that provides users with a mobile phone application capable of offering care solutions for elderly and physical disabled persons or patients. The system enables individuals to obtain jobs, care rides, ambulance services, wheelchair and gurney requests, and partnerships with pharmacies for medication deliveries. Accordingly, the present disclosure makes specific reference thereto. Nonetheless, it is to be appreciated that aspects of the present invention are also equally applicable to other like applications, devices, and methods of manufacture.

### BACKGROUND

By way of background, elderly and disabled individuals require continuous and special attention and care. Especially, old age is a sensitive phase and elderly individuals need care and comfort to lead a healthy life without worries and anxiety. For providing care to both elderly and disabled individuals, currently, it is necessary for a family member or a daycare to be physically present with the individual. However, it is difficult and sometimes impossible to be physically present with the individual. Live-in care, 24-hour Home Care, and other similar solutions are not only expensive, but are not safe as well. Individuals desire new ways to improve the quality of care for seniors and physically disabled individuals.

Many times, such individuals find themselves dependent on their family members for their expenses and even though, they want to work, they find it difficult to secure job opportunities. Further, it is difficult for elderly and disabled individuals to physically go to doctors for treatment and consultations. Often, there is a requirement of a wheelchair, a gurney and the like, which are not readily available. Individuals desire a system and method to overcome shortcomings and to improve quality of life for such individuals.

Therefore, there exists a long-felt need in the art for a system that improves quality of life of elderly and disabled individuals. There is also a long-felt need in the art for a system that reduces or obviates continuous physical presence of family members and caretakers for elderly and disabled individuals. Additionally, there is a long-felt need in the art for a system that enables elderly and disabled individuals to remotely consult doctors for treatment. Moreover, there is a long-felt need in the art for a system that provides elderly and disabled individuals job opportunities to work and earn. Further, there is a long-felt need in the art for a system that improves quality of care for elderly and disabled individuals at the touch of a button. Finally, there is a long-felt need in the art for a system that enables individuals to obtain jobs, care rides, ambulance services, wheelchair and gurney requests, and partnerships with pharmacies for medication deliveries.

The subject matter disclosed and claimed herein, in one embodiment thereof, comprises a system for providing care to elderly and physically disabled persons/patients. The system includes a computer-implemented application installed in a user device of the person or patient, the application configured to cause a processor to perform receiving registration information of the elderly or disabled patient, creating a profile of the person/patient, displaying a list of services to the person/patient, the services include at least booking a consultation, applying for a job, requesting an ambulance, requesting a wheelchair, and requesting a gurney, receiving a selection of one or more services from the person/patient, assigning one or more of a care giver, doctor, and pharmacy, wherein assignment is done based on current location of the patient. The application enables the person or patient to apply for a job and also enables a video interview for obtaining a job through the system.

In this manner, the elderly and disabled individual care system of the present invention accomplishes all of the forgoing objectives and provides users with a system that enables care seeking elderly and disabled individuals to use a smartphone application installed in the devices to obtain jobs, care rides, ambulance services, wheelchair and gurney requests, and partnerships with pharmacies for medication deliveries. The system improves quality of care at the touch of a button, thereby reducing dependencies on other family members and friends.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a system for providing care access or support to elderly and physically disabled persons or patients. The system includes a computer-implemented application installed in a user device, the application configured to cause a processor to perform receiving registration information of an elderly or disabled person/patient; creating a profile of the person/patient; displaying a list of services to the person/patient, the services include at least booking a consultation, requesting and scheduling a care visit, requesting and scheduling a care phone call, applying for a job, requesting an ambulance, requesting a wheelchair, and requesting a gurney; receiving a selection of one or more services from the person/patient; and, assigning one or more of a care giver, doctor, and/or pharmacy, wherein assignment is done based on current location of the person/patient.

In yet another embodiment, a computer-implemented method for improving quality of care for elderly and physically disabled patients is described. The method includes the steps of registering the patient as a care seeker; creating a profile of the patient; receiving a request from the patient; assigning a doctor for the patient; initiating a video call between the patient and the doctor; debiting a registered account of the patient; and, crediting registered account of the doctor.

The subject matter disclosed and claimed herein, in one embodiment thereof, comprises a computer-implemented method for providing jobs to elderly and physically disabled persons/patients. The method includes the steps of creating a profile of the person/patient, displaying a list of jobs, the jobs are selected based on current location and profile of the person/patient, receiving a selection of one or more jobs from the person/patient, arranging a video call for an interview for the job, and initiating the video call between the person/patient and the employer.

In yet another embodiment, a computer-implemented method of offering care solutions for elderly and physically disabled patients is described. The method comprising the steps of registering a patient as a care seeker, registering another user as a care giver, assigning the care giver to fulfil a request of the care seeker, wherein the care giver is selected based on close proximity of the care giver with the location of the care seeker, and providing service to the care seeker.

In yet another embodiment, the service includes at least one of care rides, ambulance services, and wheelchair and gurney requests.

In yet another embodiment, the care seeker can contact a pharmacy for medication.

Numerous benefits and advantages of this invention will become apparent to those skilled in the art to which it pertains upon reading and understanding of the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a schematic view of one potential embodiment of a medical care solution system of the present invention in accordance with the disclosed architecture;

FIG. 2 illustrates a block diagram showing components of the server system used in the medical care system of the present invention in accordance with the disclosed architecture;

FIG. 3 illustrates an exemplary first user interface displayed by a SpeedCare mobile application of the present invention in accordance with the disclosed architecture;

FIG. 4 illustrates another exemplary user interface showing the information required to be input by a user to be registered as a care seeker in accordance with the disclosed architecture;

FIG. 5 illustrates a flow diagram depicting a process of registering a care seeker and displaying relevant services offered by the medical care system of the present invention in accordance with the disclosed architecture;

FIG. 6 illustrates a flow diagram depicting the registration process of a care giver in the system of the present invention in accordance with the disclosed architecture;

FIG. 7 illustrates an exemplary user interface of the mobile application of the present invention displaying different service options offered to a care seeker in accordance with the disclosed architecture;

FIG. 8 illustrates an exemplary user interface showing detection of current location of a care seeker by the SpeedCare application of the present invention in accordance with the disclosed architecture;

FIG. 9 illustrates a flow diagram depicting a process of assigning and notifying of a care giver for servicing a request of a care seeker in accordance with the disclosed architecture;

FIG. 10 illustrates a flow diagram showing a process of registering a pharmacy and assigning pharmacy to a care seeker’s request in accordance with the disclosed architecture;

FIG. 11 illustrates a flow diagram depicting a process offer assigning a doctor and providing a consultation to a care seeker using the system of the present invention in accordance with the disclosed architecture; and

FIG. 12 illustrates an exemplary user device that is used for installing the SpeedCare mobile application of the present invention in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a system that improves quality of life of elderly and disabled individuals. There is also a long-felt need in the art for a system that reduces continuous physical presence of family members and caretaker for elderly and disabled individuals. Additionally, there is a long-felt need in the art for a system that enables elderly and disabled individuals to remotely consult doctors for treatment. Moreover, there is a long-felt need in the art for a system that provides elderly and disabled individuals jobs opportunities to work and earn. Further, there is a long-felt need in the art for a system that improves quality of care for elderly and disabled individuals at the touch of a button. Finally, there is a long-felt need in the art for a system that enables individuals to obtain jobs, care rides, ambulance services, care visits, wheelchair and gurney requests, and partnerships with pharmacies for medication deliveries.

The present invention, in one exemplary embodiment, is a computer-implemented method for improving quality of care for elderly and physically disabled persons/patients. The method includes the steps of registering the patient as a care seeker, creating a profile of the person/patient, receiving a request from the person/patient, assigning a doctor for the person/patient, initiating a video call between the person/patient and the doctor, debiting a registered account of the person/patient, and crediting the registered account of the doctor.

In the present disclosure, the “care seeker” is comprehended as elderly and physical disabled patients who want to avail the services offered by the system. The “care giver” are users who provide services to the care seekers.

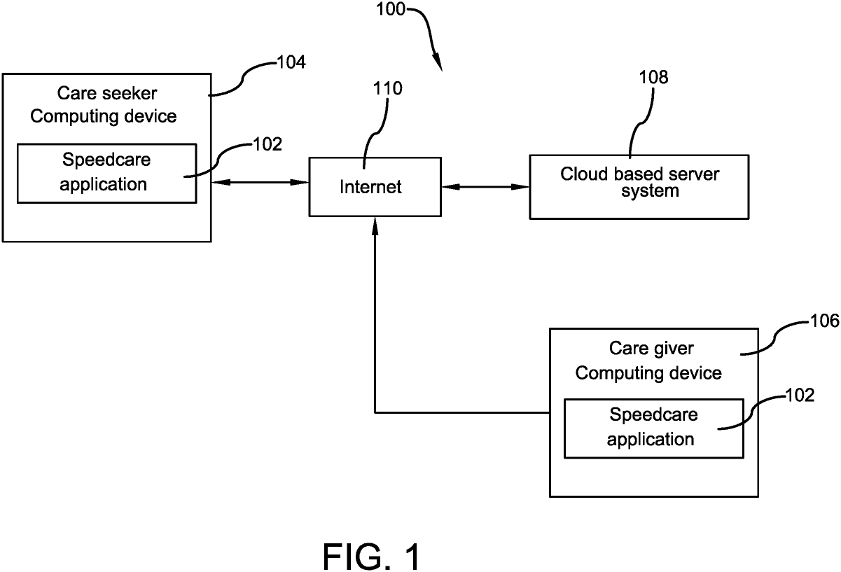

Referring initially to the drawings, FIG. 1 illustrates a schematic view of one potential embodiment of medical care solution system of the present invention in accordance with the disclosed architecture. The system 100 functions using computer-implemented SpeedCare application 102 installed in user devices such as smartphones or any other handheld electronic devices. More specifically, the system 100 enables installation of the SpeedCare application 102 in a care seeker user or computing devices 104 and in a care giver computing devices 106. The computing devices 104, 106 can be same in terms of configuration and are shown separately to show distinction between the care seeker devices and care giver devices. The system 100 is configured to enable users to register as care seekers for requesting wheelchairs, gurney, pharmacy, medicines, care visits, care calls, jobs and more, offered by the system 100. The users can also be registered as care givers and are configured to provide requested services to the care seekers.

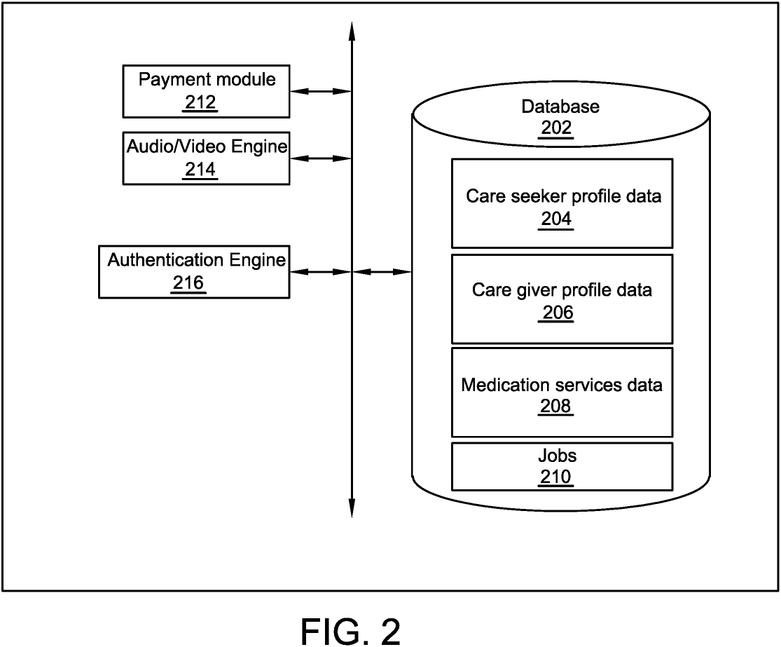

The system 100 has a cloud server 108 for providing necessary functionalities and user interfaces in the SpeedCare application 102 and details of the server 108 are described in FIG. 2. The user devices 104, 106 and the server 108 are communicatively coupled to each other via a communication network 110 such as the Internet. The user devices 104, 106 are capable of facilitating a user to input data, receive data, and/or transmit data within the system 100 using the application 102. It will be apparent to a person of ordinary skill in the art that the care seekers may be any personnel using the system 100 for connecting with the care givers without deviating from the scope of the disclosure. Examples of the user devices 104, 106 may include, but are not limited to, a desktop, a notebook, a laptop, a handheld computer, a touch sensitive device, a computing device, a smart-phone, and/or a smart watch.

The server 108 is a network of computers, a software framework, or a combination thereof, that may provide a generalized approach to create the server implementation. The server 108 can be realized through various web-based technologies such as, but not limited to, a Java web-framework, a .NET framework, a personal home page (PHP) framework, or any web-application framework. The server 108 may be maintained by a storage facility management authority or a third-party entity that facilitates service enablement and resource allocation operations of the system 100.

The communication network 110 may include suitable logic, circuitry, and interfaces that may be configured to provide a plurality of network ports, a plurality of communication channels for transmission and reception of data related to operations of various entities (such as the user devices 104, 106 and the server 108) of the system 100. Each network port may correspond to a virtual address (or a physical machine address) for transmission and reception of the communication data. For example, the virtual address may be an Internet Protocol Version 4 (IPV4) (or an IPV6 address) and the physical address may be a Media Access Control (MAC) address. The communication network 106 may be associated with an application layer for implementation of communication protocols. The communication data may be transmitted or received, via the communication protocols. Examples of the communication protocols may include, but are not limited to, Hypertext Transfer Protocol (HTTP), File Transfer Protocol (FTP), Simple Mail Transfer Protocol (SMTP), Domain Network System (DNS) protocol, Common Management Interface Protocol (CMIP), Transmission Control Protocol and Internet Protocol (TCP/IP), User Datagram Protocol (UDP), Long Term Evolution (LTE) communication protocols, or any combination thereof.

FIG. 2 illustrates a block diagram showing components of the server system 108 used in the medical care system of the present invention in accordance with the disclosed architecture. The server 108 has a database 202 that includes a plurality of data used in authentication of users of the system 100. More specifically, a care seeker profile data module 204 is configured to store profile data of all the care seekers registered in the system. The information includes name, location, age, payment information, and more in an encrypted form in the database enabling the system to authenticate the care seekers. A care giver profile data module 206 is configured to store profile data of all the care givers registered in the system. The information stored in the database includes at least name, age, authentication information, payment information, and more in an encrypted form enabling the system to authenticate the care givers. In the preferred embodiment of the present invention, all the users in the system are authenticated and verified before getting registered in the system thereby maintaining the safety and security of all the registered users. A medication services data module 208 is configured to store pharmacy and doctor data registered in the system. The system is configured to assign a pharmacy and a medical practitioner based on requirement and request of a care seeker. The job data module 210 is configured to store active jobs listed in the system 100 enabling elderly and physically disabled individuals to apply for the jobs.

A payment module 212 is configured to process payments involved in the system 100 when the care seekers request the services and the care givers, pharmacies, doctors, and more provide the services. An audio/video engine 214 is configured to integrate audio and video call functionality in the application 102 which can be used for interacting in telephonic interviews for jobs, to provide medical consultations (i.e., care visits/calls), and more. An authentication engine 216 is configured to authenticate and perform background checks of the users registering in the system 100. In some embodiments, authentication engine 216 may couple with third-party servers and databases for an effective authorization and authentication of users of the system 100.

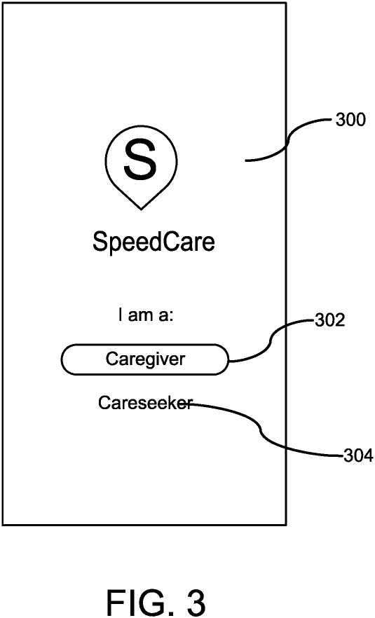

FIG. 3 illustrates an exemplary first user interface displayed by the SpeedCare mobile application of the present invention in accordance with the disclosed architecture. The interface 300 is the first screen shown to a user after installation of the application 102 and enables a user to register either as a care giver or a care seeker depending on the requirements of a user. Accordingly, a user can select the care giver tab 302 on the interface 300 for registering as a care giver to provide services through the system 100. The user can also select the care seeker tab 304 for registering as a care seeker to avail the services offered by the system 100.

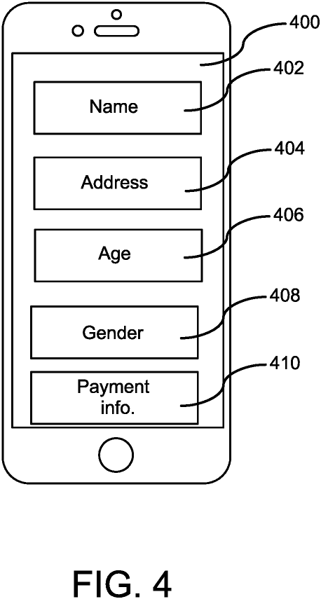

FIG. 4 illustrates another exemplary user interface showing the information required to be input by a user to be registered as a care seeker in accordance with the disclosed architecture. For initial registration as a care seeker, the application 102 through the user interface 400 requires a user to sign up. A name box 402 which may include first name, middle name, last name enables the user to input the name, an address box 404 is used for receiving address of the user. The address is used for providing relevant services and providing relevant care givers to the user.

As the system 100 is preferably designed for elderly individuals, therefore, an age box 406 is used for receiving age of the user. It should be noted that system 100 can also be used by individuals of all ages and genders, wherein the gender box 408 is used for inputting gender information. For availing services offered by the system 100 and to pay the care givers and for the medicines, a payment method can be saved in the application 102 by the user using the payment information box 410. The system 100 uses the stored payment information for processing payments and can include credit card, debit card, gift card, or any other payment methods.

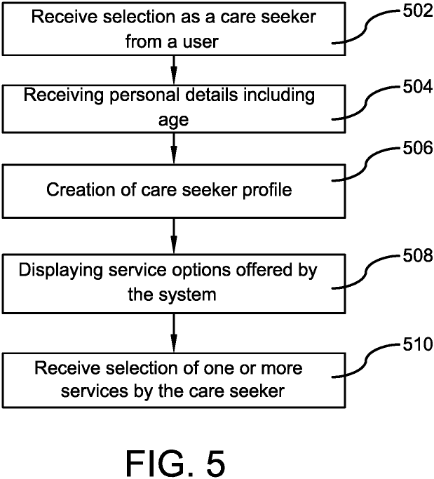

FIG. 5 illustrates a flow diagram depicting a process of registering a care seeker and displaying relevant services offered by the medical care system of the present invention in accordance with the disclosed architecture. Initially, a user selects registration as a care seeker using the application as illustrated in FIG. 3 (Step 502). Next, the application receives personal details and payment details from the care seeker using the interface of the application (Step 504). Then, the system 100 creates the care seeker profile and stores same in the database of the system (Step 506).

Upon successful registration, different services offered by the system for the care seeker are displayed (Step 508). It should be noted that in some embodiments of the present invention, the system can display different services to different care seekers based on their past services, preferences, behavior, and more. Finally, selection of one or more services are received by the system enabling the system to provide the selected service to the care seeker (Step 510).

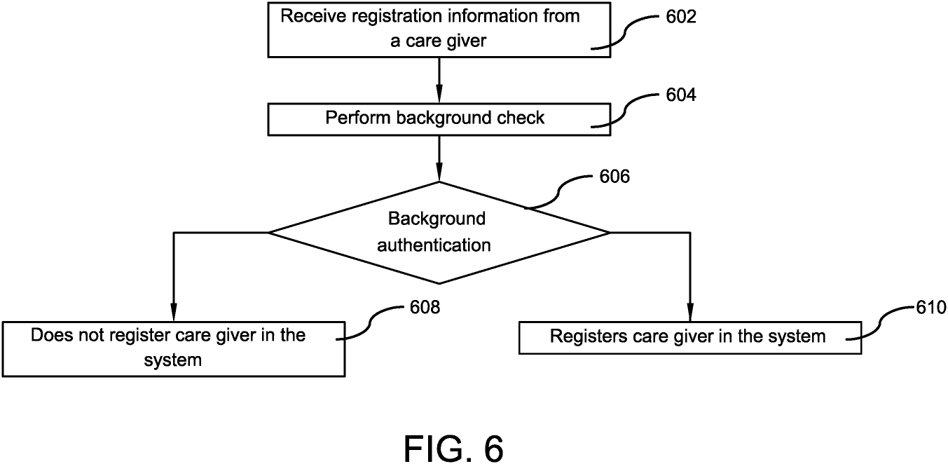

FIG. 6 illustrates a flow diagram depicting a flow diagram showing registration process of a care giver in the system of the present invention in accordance with the disclosed architecture. Initially, registration information including, but not limited to, name, age, SSN, location, and more is received from a user intending to register as a care giver (Step 602). Next, before registration, a background check is performed of the user (Step 604). During the background check, third-party agencies may be used by the system for authentication and further, documents uploaded by the user are also cross-checked for verification.

At the next step 606, the background authentication is performed by the system 100. The authentication is automatic and a notification of successful or unsuccessful authentication is provided to the user registering as a care giver. In case, the authentication is not successful, then, the care giver is not registered in the system for maintaining security of the system and registered care seekers (Step 608). In case the background verification is successful, then, the user is registered as a care giver in the system (Step 610).

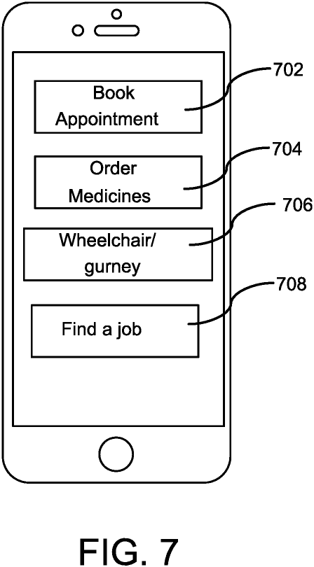

FIG. 7 illustrates an exemplary user interface of the mobile application of the present invention displaying different service options offered to a care seeker in accordance with the disclosed architecture. A book appointment option 702 enables a care seeker to have an appointment or visit with a doctor or a medical practitioner for treatment or medical consultation. The application enables a care seeker to book an appointment or visit for a physical consultation or a consultation through audio or video call. Further, the system has a plurality of registered medical practitioners and are assigned to a care seeker based on the preferences and requirement of the care seeker. Nearby and recommended medical practitioner is automatically provided to the care seeker. The video call functionality can also be used for interviewing prospective applicants for jobs offered through the system 100. The elderly individuals and physically disabled individuals can easily be interviewed using the integrated video and audio call functionality without physically going anywhere.

An order medicine option 704 enables a care seeker to order medicines from registered pharmacies. A care giver is assigned for the care seeker’s request and the care giver provides the medicines at the current location of the care seeker where in the location is detected using the GPS of the user device of the care seeker or the location manually input by the care seeker.

The system 100 of the present invention enables a care seeker to request for a wheelchair or gurney using the wheelchair/gurney/ambulance option 706. A care giver is assigned for the request and the requested wheelchair, ambulance, or gurney is provided at the located or provided address of the care seeker. An advantage of the present invention is that it enables a care seeker to find and request for jobs enabling them to work and find jobs as per their preferences and locations. More specifically, using the “Find a job” option 708, a care seeker can find a list of nearby jobs and the list can be personalized based on the preferences and recommendations of the care seeker. It is contemplated that many other features that are useful for elderly and physically limited individuals can be included in the system 100 and can be offered to the care seeker through the medical care application of the present invention.

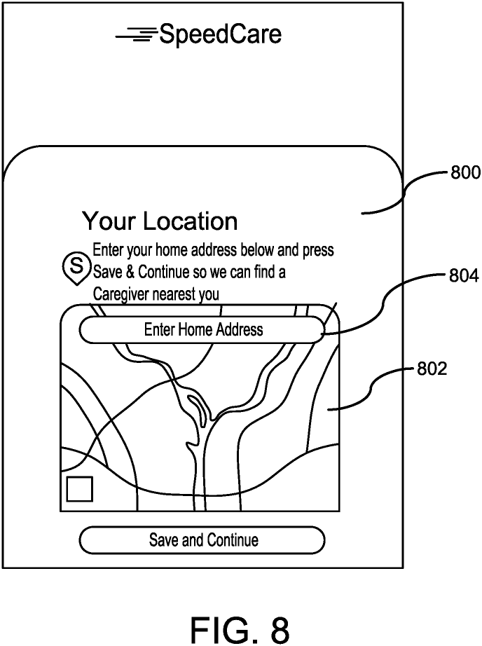

FIG. 8 illustrates an exemplary user interface showing detection of current location of a care seeker by the SpeedCare application of the present invention in accordance with the disclosed architecture. The system 100 of the present invention enables a care seeker to request different services offered by the system as illustrated in FIG. 7. A care giver whose location is near to the location of the care seeker is automatically or preferentially assigned by the system 100 for providing services to the care seeker. The location of the care seeker can be detected automatically using the GPS functionality of the user device in which the care seeker application is installed or can also be detected manually wherein the care seeker inputs the current location using text box 804 where the services are required by the care seeker. Location of the care seeker is displayed in the form of a digital map 802 which is displayed on the user interface 800 of the application 102. In some embodiments of the present invention, the location of the assigned care giver is also displayed on the digital map enabling the care seeker to know the real-time update of the location of the care seeker.

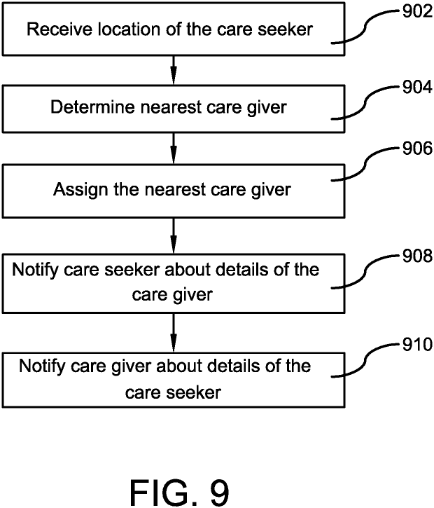

FIG. 9 illustrates a flow diagram depicting a process of assigning and notifying of a care giver for servicing a request of a care seeker in accordance with the disclosed architecture. Initially, current location of care seeker is detected and received by the SpeedCare application installed in the user device of the care seeker (Step 902). The location can be detected using GPS functionality or manual input of the care seeker. Then, the nearest available care giver is automatically determined and selected for servicing the request by the system (Step 904). Thereafter, the determined care giver is assigned for servicing the request of the care seeker (Step 906).

For effective communication, details of the care giver are notified to the care seeker in the form of a notification wherein the notification can be in the form of audio or text (Step 908). Real time updates of the service are also provided to the care seeker by the system 100. Finally, a notification of details of the care seeker is provided to the assigned care giver wherein details of the services are also included in the notification provided to the care giver (Step 910).

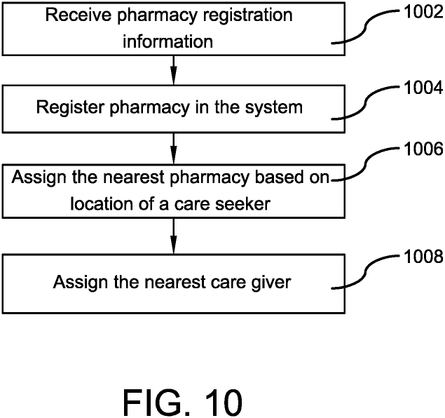

FIG. 10 illustrates a flow diagram showing process registering a pharmacy and assigning pharmacy to a care seeker request in accordance with the disclosed architecture. Initially, the system receives registration information from a pharmacy wherein the information includes at least name, location, different medicines available in the pharmacy and more (Step 1002). Then, after a background check, the pharmacy is registered in the system and information of the pharmacy is updated in the database of the system (Step 1004). Thereafter, based on request, profile, and location of a care seeker, the nearest pharmacy is assigned for a medicine request of the care seeker (Step 1006) and finally, the nearest care giver is assigned and provides the medicine to the care seeker on the detected location of the care seeker (Step 1008).

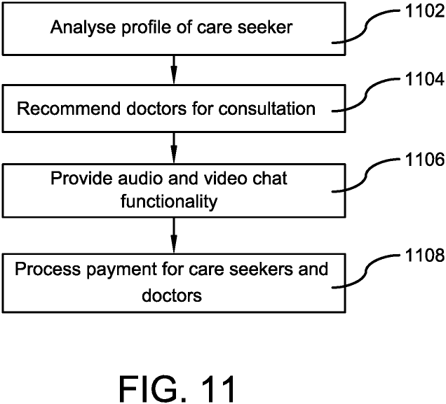

FIG. 11 illustrates a flow diagram depicting a process assigning a doctor and providing a consultation to a care seeker using the system of the present invention in accordance with the disclosed architecture. Initially, the system analyzes the profile of a care seeker where in past orders, preferences, profile details, location and more of the care seeker are analyzed (Step 1102). Then, nearby doctors or medical practitioners are recommended and assigned for consultation to the care seeker (Step 1104). Then, based on preference of the care seeker, either physical consultation or audio/video consultation are provided by the system 100 through the application installed in the user device of the care seeker (Step 1106). Finally, payment processing is done and an amount is debited from the care seeker and is credited to the care giver/doctor (Step 1108).

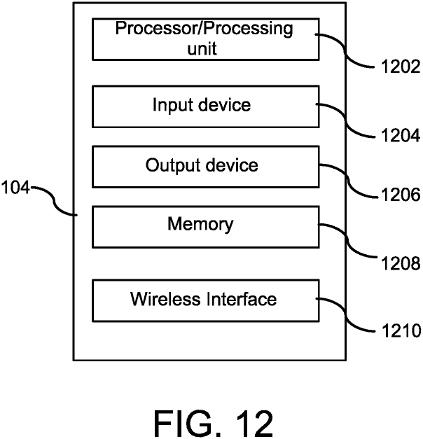

FIG. 12 illustrates an exemplary user device 104 that is used for installing the SpeedCare mobile application 102 of the present invention in accordance with the disclosed architecture. The processing unit 1202 may include suitable logic, instructions, circuitry, interfaces, and/or codes for executing various operations, such as the operations associated with the user device 104, or the like. The processing unit 1202 may be configured to control one or more operations executed by the user device 104 in response to the input received at the user device 104 from the user. The processor 1202 executes the computer readable instructions stored in the application 102. Examples of the processing unit 1202 may include, but are not limited to, an application-specific integrated circuit (ASIC) processor, a reduced instruction set computing (RISC) processor, a complex instruction set computing (CISC) processor, a field-programmable gate array (FPGA), a Programmable Logic Control unit (PLC), and the like. Embodiments of the present disclosure are intended to include or otherwise cover any type of the processing unit 1202 including known, related art, and/or later developed processing units. The user device 104 can further include one or more computer executable applications configured to be executed by the processing unit 1202. The one or more computer executable applications may include suitable logic, instructions, and/or codes for executing various operations. The one or more computer executable applications may be stored in the memory 1208. The one or more computer executable applications includes the application 102.

The user device 104 includes input device(s) 1204 such as a touch input device, voice input device, etc. for entering data and information. Preferably, the touch interface of the user device 104 is used as the input and various buttons/tabs shown on the SpeedCare application 102 are pressed or clicked by the user. Other input devices such as camera and microphone are used during video chatting by the user. The display of the user device 104 also acts as the output device 1206 for displaying various contents (i.e., text, images, videos, icons, and / or symbols, etc.) to the user. The display can include a touch screen, and may receive, for example, a touch, gesture, proximity, or hovering input using an electronic pen or a part of a user’s body.

Electronic device 104 has memory 1208 used for storing programs (sequences of instructions) or data (e.g., program state information) on a temporary or permanent basis for use in the computer system. Memory 1208 can be configured for short-term storage of information as volatile memory and therefore not retain stored contents if powered off. Examples of volatile memories include random access memories (RAM), dynamic random-access memories (DRAM), static random-access memories (SRAM), and other forms of volatile memories known in the art. The processor 1202, in combination with one or more of memory 1208, input device(s) 1204, output device(s) 1206 is utilized to provide users to execute instructions on the application 102. The connection to a network is provided by wireless interface 1210.

The wireless interface 1210 enables the user device 104 to communicate with the server system and other components of the system 100 over the communication network, according to embodiments of the present disclosure. Examples of the communication interface 1210 may include, but are not limited to, a modem, a network interface such as an Ethernet card, a communication port, and/or a Personal Computer Memory Card International Association (PCMCIA) slot and card, an antenna, a radio frequency (RF) transceiver, one or more amplifiers, a tuner, one or more oscillators, a digital signal processor, a coder-decoder (CODEC) chipset, a subscriber identity module (SIM) card, and a local buffer circuit. It will be apparent to a person of ordinary skill in the art that the communication interface 1210 may include any device and/or apparatus capable of providing wireless or wired communications between the user device 104 and the server.

Certain terms are used throughout the following description and claims to refer to particular features or components. As one skilled in the art will appreciate, different persons may refer to the same feature or component by different names. This document does not intend to distinguish between components or features that differ in name, but not structure or function. As used herein “medical care system”, “medical care system for elderly and physically disable patients”, and “system” are interchangeable and refer to the medical care system 100 of the present invention.

Notwithstanding the forgoing, the medical care system 100 of the present invention can be of any suitable configuration as is known in the art without affecting the overall concept of the invention, provided that it accomplishes the above-stated objectives. One of ordinary skill in the art will appreciate that the medical care system 100 as shown in the FIGS. are for illustrative purposes only, and that many other configurations, components, and design of the medical care system 100 are well within the scope of the present disclosure. Although the mentioned components of the medical care system 100 are important design parameters for user convenience, the medical care system 100 may contain any additional components that ensures optimal performance during use and/or that suits the user’s needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A computer-implemented electronic device application comprising:
a computer-implemented system having an application;
a communication network; and
a server to connect a care giver through a care giver computing device and a care seeker through a care seeker computing device;
wherein said application configured to enable users to register as said care giver, wherein said care giver provides a plurality of services for said care seeker;
wherein said application configured to enable users to register as said care seeker, wherein said care seeker needs said plurality of services provided from said care giver;
wherein said communication network including an Internet connection;
wherein said care giver computing device and said care seeker computing device including a user’s input data and the user’s transmit data;
wherein said electronic device selected from a group consisting of a smartphone, a desktop computer, a notebook computer, a laptop computer, a handheld computer, a touch sensitive device, a computing device, and a smart watch;
wherein said server connects a network of computers and said server is selected from a group consisting of a Java web-framework, a .NET framework, a personal home page (PHP) framework, and a web-application framework;
wherein said server is a cloud server for interfacing said care giver computing device and said care seeker computing device;
wherein said communication network having a plurality of network ports and a plurality of communication channels for transmission and reception of data between said care giver computing device, said server, and said care seeker computing device;
wherein said data having a care giver profile module for storing a care giver profile;
wherein said care giver profile includes information selected from a group consisting of a name, an age, a SSN, a location, an authentication information, a payment information, and a listing of said plurality of services provided in an encrypted form for authenticating said care givers;
wherein said data having a care seeker profile module for storing said care seeker profile; and
further wherein said care seeker profile includes information selected from a group consisting of a name, a SSN, a location, an age, an authentication information, a payment information, and a listing of said plurality of services needed in an encrypted form for authenticating said care seekers.

2. The computer-implemented electronic device application of claim 1, wherein said plurality of services are selected from a group consisting of a wheelchair, a gurney, a pharmacy, a medicine, a care visit, a care call, and a job.

3. The computer-implemented electronic device application of claim 2, wherein each of said plurality of network ports corresponding to a virtual address of one of said care giver computing devices and one of said care seeker computing devices for said transmission and said reception of said data.

4. The computer-implemented electronic device application of claim 3, wherein said virtual address selected from a group consisting of an Internet Protocol Version 4 address and an Internet Protocol Version 6 address.

5. The computer-implemented electronic device application of claim 4, wherein said virtual address is a physical address, including a Media Access Control (MAC) address.

6. The computer-implemented electronic device application of claim 5, wherein said communication network including a communication protocol for said transmitting and said receiving said data, further wherein said communication protocol selected from a group consisting of a Hypertext Transfer Protocol (HTTP), a File Transfer Protocol (FTP), a Simple Mail Transfer Protocol (SMTP), a Domain Network System (DNS) protocol, a Common Management Interface Protocol (CMIP), a Transmission Control Protocol and Internet Protocol (TCP/IP), a User Datagram Protocol (UDP), and a Long Term Evolution (LTE).

7. The computer-implemented electronic device application of claim 6, wherein said server having a database including a plurality of data for authenticating the users of said computer-implemented system.

8. The computer-implemented electronic device application of claim 7 further comprising a medication services data module for storing a store pharmacy registry and a doctor data registry in said application.

9. The computer-implemented electronic device application of claim 8, wherein said medication services data module assigns a pharmacy and a medical practitioner to said care seeker based on said care seeker profile.

10. The computer-implemented electronic device application of claim 9 further comprising a job data module for storing active jobs in said application.

11. The computer-implemented electronic device application of claim 10, wherein said job data module connects said active jobs to said care seeker based on said care seeker profile.

12. The computer-implemented electronic device application of claim 11 further comprising a payment module for processing payments between said care givers and said care seekers.

13. The computer-implemented electronic device application of claim 12 further comprising an audio/video module to integrate an audio call and a video call in said application for the connecting between said care givers and said care seekers.

14. A computer-implemented electronic device application comprising:
a computer-implemented system having an application;
a communication network;
a server to connect a care giver through a care giver computing device and a care seeker through a care seeker computing device;
wherein said application configured to enable users to register as said care giver, wherein said care giver provides a plurality of services for said care seeker;
wherein said application configured to enable users to register as said care seeker, wherein said care seeker needs said plurality of services provided from said care giver;
wherein said communication network including an Internet connection;
wherein said care giver computing device and said care seeker computing device, including a user’s input data and the user’s transmit data;
wherein said electronic device selected from a group consisting of a desktop computer, a smartphone, a notebook computer, a laptop computer, a handheld computer, a touch sensitive device, a computing device, and a smart watch;
wherein said server connects a network of computers and said server is selected from a group consisting of a Java web-framework, a .NET framework, a personal home page (PHP) framework, and a web-application framework;
wherein said server is a cloud server for interfacing said care giver computing device and said care seeker computing device;
wherein said communication network having a plurality of network ports and a plurality of communication channels for transmission and reception of data between said care giver computing device, said server, and said care seeker computing device;
wherein said data having a care giver profile module for storing a care giver profile;
wherein said care giver profile includes information selected from a group consisting of a name, an age, a SSN, a location, an authentication information, a payment information, and a listing of said plurality of services provided in an encrypted form for authenticating said care givers;
wherein said data having a care seeker profile module for storing said care seeker profile;
wherein said care seeker profile includes information selected from a group consisting of a name, an age, a SSN, a location, an authentication information, a payment information, and a listing of said plurality of services needed in an encrypted form for authenticating said care seekers;
a payment module for processing payments between said care givers and said care seekers;
an audio/video module to integrate an audio call and a video call in said application for the connecting between said care givers and said care seekers; and
a medication services data module for storing a store pharmacy registry and a doctor data registry in said application;
wherein said medication services data module assigns a pharmacy and a medical practitioner to said care seeker based on said care seeker profile.

15. The computer-implemented electronic device application of claim 14, wherein said plurality of services are selected from a group consisting of a wheelchair, a gurney, a pharmacy, a medicine, a care visit, a care call, and a job.

16. The computer-implemented electronic device application of claim 15, wherein each of said plurality of network ports corresponding to a virtual address of one of said care giver computing devices and one of said care seeker computing devices for said transmission and said reception of said data.

17. A computer-implemented electronic device application comprising:
a computer-implemented system having an application;
a communication network;
a server to connect a care giver through a care giver computing device and a care seeker through a care seeker computing device;
wherein said application configured to enable users to register as said care giver, wherein said care giver provides a plurality of services for said care seeker;
wherein said application configured to enable users to register as said care seeker, wherein said care seeker needs said plurality of services provided from said care giver;
wherein said communication network, including an Internet connection;
wherein said care giver computing device and said care seeker computing device, including a user’s input data and the user’s transmit data;
wherein said electronic device selected from a group consisting of a desktop computer, a smartphone, a notebook computer, a laptop computer, a handheld computer, a touch sensitive device, a computing device, and a smart watch;
wherein said server connects a network of computers and said server is selected from a group consisting of a Java web-framework, a .NET framework, a personal home page (PHP) framework, and a web-application framework;
wherein said server is a cloud server for interfacing said care giver computing device and said care seeker computing device;
wherein said communication network having a plurality of network ports and a plurality of communication channels for transmission and reception of data between said care giver computing device, said server, and said care seeker computing device;
wherein said data having a care giver profile module for storing a care giver profile;
wherein said care giver profile includes information selected from a group consisting of a name, an age, a SSN, a location, an authentication information, a payment information, and a listing of said plurality of services provided in an encrypted form for authenticating said care givers;
wherein said data having a care seeker profile module for storing said care seeker profile;
wherein said care seeker profile includes information selected from a group consisting of a name, an age, a SSN, a location, an authentication information, a payment information, and a listing of said plurality of services needed in an encrypted form for authenticating said care seekers;
a payment module for processing payments between said care givers and said care seekers;
an audio/video module to integrate an audio call and a video call in said application for the connecting between said care givers and said care seekers; and
a medication services data module for storing a store pharmacy registry and a doctor data registry in said application;
wherein said medication services data module assigns a pharmacy and a medical practitioner to said care seeker based on said care seeker profile; and
further wherein said care giver profile including a background check of said care giver.

18. The computer-implemented electronic device application of claim 14, wherein said plurality of services are selected from a group consisting of a wheelchair, a gurney, a pharmacy, a medicine, a care visit, a care call, and a job.

19. The computer-implemented electronic device application of claim 15, wherein each of said plurality of network ports corresponding to a virtual address of one of said care giver computing devices and one of said care seeker computing devices for said transmission and said reception of said data.

20. The computer-implemented electronic device application of claim 19, wherein said background check including a background authentication, and further wherein said background authentication is either successful or unsuccessful for said care giver.
